(19) United States
(12) Patent Application Publication
(10) Pub. No.: US 2008/0287830 A1
(43) Pub. Date: Nov. 20, 2008

(54) Apparatus and Method for Portable Blood Cell Counting

(76) Inventor: John G. Voeller, Leawood, KS (US)

Correspondence Address: FOX ROTHSCHILD LLP, 1101 MARKET STREET, SUITE 2600, PHILADELPHIA, PA, 19107-2950, US

(21) Appl. No.: 12/095,790

(22) PCT Filed: Dec. 1, 2006
(86) PCT No.: PCT/US2006/045881

§ 371 Date: Jun. 2, 2008

Related U.S. Application Data
(60) Provisional application No. 60/741,690, filed on Dec. 2, 2005.

(51) Int. Cl. A61B 5/157

(52) U.S. Cl. 600/573

(57) ABSTRACT

The invention relates to a novel self-contained apparatus and method for portable and rapid counting of blood cells in a sample drawn from a subject in need of determining his or her blood cell counts. The apparatus may be about the size of a credit card, and rapidly determines a subject's blood cell counts.

### FIELD OF THE INVENTION

The invention relates to a novel apparatus and method for portable and rapid counting of blood cells in a sample drawn from a mammal, preferably a human.

### BACKGROUND OF THE INVENTION

A person's blood cell count is one of the most commonly tested parameters in clinical laboratories. For white blood cells, a typical laboratory can provide a white blood cell count from a sample normally requiring from about 5-10 ml of whole blood. The blood is mixed with reagents, then counted in a flow cell. Such flow cells are expensive and require professionally trained personnel in order to run in a clinical laboratory.

Rapid determination of white blood cell counts may be critical in a physician's office, in a hospital, or at home where any detrimental hematologic effects of therapeutic regimes on white blood cell count must be determined. Many medications are known to have an effect on white blood cell count, therefore requiring regular monitoring of white blood cell count (e.g., Clozapine). Further, chemotherapy and other patients must have their red and white blood cell counts closely monitored. The ability to monitor large populations following potential nuclear radiation accidents or terrorist attacks also makes clear the need for rapid and inexpensive determinations of blood cell counts.

The promise of microfluidics has been realized in some contexts, but not in the ability to provide rapid and inexpensive blood cell counts. For example, the ABO card (Micronics Inc.) allows for the rapid determination of blood type using a credit card sized device containing all necessary reagents and readouts. All that is required is a blood sample. See, e.g., U.S. Pat. Nos. 5,932,100; 6,136,272; and 6,488,896, each of which are specifically incorporated herein in their entirety.

As stated by Clayton in Nature Methods 2, 621-627 (2005), microfluidics now appears ready to transform traditional assay systems in academia and biotech as well as in big pharma and hospitals, with devices labeled as ‘pinhead petri dishes’ and ‘Lab-on-a-chip.’ Microfluidic devices have been known in the art for only a few years, beginning primarily with such lab-on-a-chip devices requiring samples to be introduced into the device in a highly specific form, such as premixed in a homogenous reagent mixture. A review in 2003 concluded that while many microfluidic devices were in active development, integration of all laboratory functions on a chip, and the commercialization of truly hand-held, easy to use microfluidic instruments have yet to be fulfilled. Weigl, Advanced Drug Delivery Reviews, 55 (2003) 349-377, specifically incorporated herein by reference in its entirety. Weigl describes several microfluidics devices, however, none are capable of reading out a blood cell count on the device itself, and particularly not in a matter of minutes.

U.S. Pat. No. 6,709,868 discloses a method and device for counting white blood cells in a sample based on indirect measurement through dye labeled substrates activated by enzymes present on white blood cells. The results are read semi-quantitatively by eye by comparing to a color chart, and may be quantitatively by a reflectometer. The device is not capable of counting red blood cells, nor is it sufficiently portable as is the device of the invention, which does not rely on chemical reactions with the blood sample, and does not require separate reader devices.

The art is therefore in need of an apparatus and associated methods for the rapid and inexpensive determination of blood cell counts.

### SUMMARY OF THE INVENTION

The present invention provides a novel apparatus and methods for portable and rapid counting of blood cells in a sample drawn from a mammal, preferably a human.

It is an object of the invention to provide an apparatus and a method of counting red blood cells, and counting white blood cells, independently or simultaneously. The apparatus and method are particularly well-suited as a health monitor for those post-operative or post-surgical procedure, as early infection detectors without laboratory work, as home monitors for infections, for emergency workers, and for chemical and biological attack effects sensing. The apparatus may be packaged in sterile containers, or sterile envelopes. The user may use his thumb, or any other digit or close vein body part.

The apparatus is self-contained, requiring no additional components or materials other than a blood sample drawn from a subject. The apparatus may range in size from about 1 inches by 2 inches by one tenth of an inch, to about the size of a standard credit card or larger, and may be carried by the end user or other personnel in need of employing the device to measure the blood cell count of another. The method of the invention comprises the steps of pressing a pad on the apparatus which comprises microneedles capable of drawing blood, applying pressure to the same or a different pad to generate a pumping force which carries the blood sample through the device where it is combined with a reagent, separated into channels which separately carry white and red blood cells respectively across a counting means comprising counting sensors. The sensors provide data to a logic chip embedded within the apparatus which outputs a signal readable on the apparatus, indicating the resultant white and red blood cell counts.

In one embodiment, the invention is a self-contained apparatus for counting blood cells comprising a substrate with an upper surface and a lower surface, the substrate comprising (a) a microneedle pad being capable of drawing a blood sample from the digit of a human subject (b) a pump capable of forcing the blood sample into a blood and reagent mixing bulb, wherein the blood sample is treated with reagents to yield a treated sample free of undesired blood components, and of desired viscosity and dilution; (c) blood cell filtration elements to filter white blood cells into a white blood cell counting channel and red blood cells into a red blood cell counting channel, the diameter of at least the distal portion of the channels being sufficient for only a single file of blood cells to occupy the counting channels, wherein the distal ends of the channels narrow to prevent the further passage of blood cells; and (d) an array of counting sensors adjacent to the blood cell counting channels, the sensors being in communication with a logic chip adapted to determine blood cell counts from the data communicated by the sensors and to display the blood cell counts on cell count indicators on the upper surface of the substrate.

In another embodiment, the apparatus further comprises at least one component selected from the group consisting of a validity indicator circuit, a clean sample indicator, a bladder seal, a bladder adhesion ring, a sample holding chamber, a flat cell battery, a solar cell, a blackflow check valve, an air accumulator bulb, air suction channels, an air suction draw tube, an air entry channel, and instructions printed or etched on the upper surface of the substrate. The substrate may comprise an upper substrate and a lower substrate, wherein the joinder of the upper and lower substrates forms the channels and chambers by complementary grooves in an interior surface of the upper and lower substrates. Alternatively, the substrate may comprise a plurality of layers laid down by thin layer deposition under computer aided design (CAD) control. Alternatively, the substrate comprises an upper substrate, a middle substrate, and a lower substrate joined together.

The pump may be manually operated by the subject successively pressing and releasing the microneedle pad. The blood sample drawn into the apparatus has a volume between 50 μl and 1 ml. The blood cell counts may be displayed on the upper surface of the substrate by LEDs.

In another embodiment, a self-contained apparatus of the invention can be used for counting just red or just white blood cells by eliminating or collecting the undesired blood cell type, and allowing only the desired cell type to pass through a single set of chambers and channels to a single counting channel, the results of which are displayed on a single LED indicator, or other indicator capable of producing a visible readout (e.g., LCD, backlit LCD, surface gas lamp, and the like).

The methods of the invention comprise methods of counting blood cells in a subject by using any of the apparatus of the invention.

These and other features of the invention are exemplified and further described in the Detailed Description of the Invention below.

### BRIEF DESCRIPTION OF THE DRAWINGS

FIG. 1 is a schematic representation depicting the outer upper substrate of an embodiment of the apparatus of the invention.

FIG. 2 is a schematic representation depicting the outer upper substrate of an embodiment of the apparatus of the invention, illustrating particularly the microneedle pad and bladder components.

FIG. 3 is a schematic representation depicting an embodiment of the apparatus of the invention, showing the upper and either central or lower substrates, and illustrating particularly the pathway for a blood sample through the apparatus, the counting sensors, the battery, and the validity indicator circuit.

FIG. 4 is a schematic representation depicting an embodiment of the apparatus of the invention, showing either the middle or lower substrate, and illustrating particularly the pathway for air through the apparatus, along with the counting sensors, battery, and validity indicator circuit.

### DETAILED DESCRIPTION OF THE INVENTION

### Apparatus for Counting Red and White Blood Cells

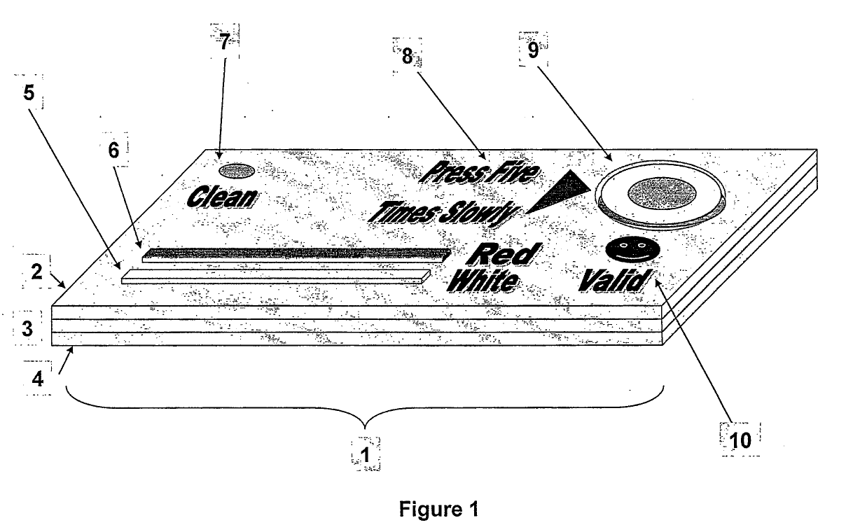

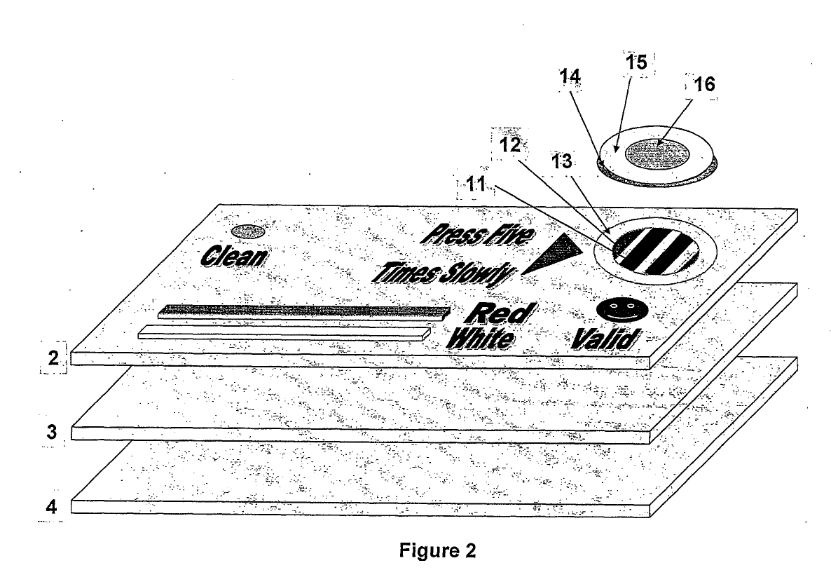

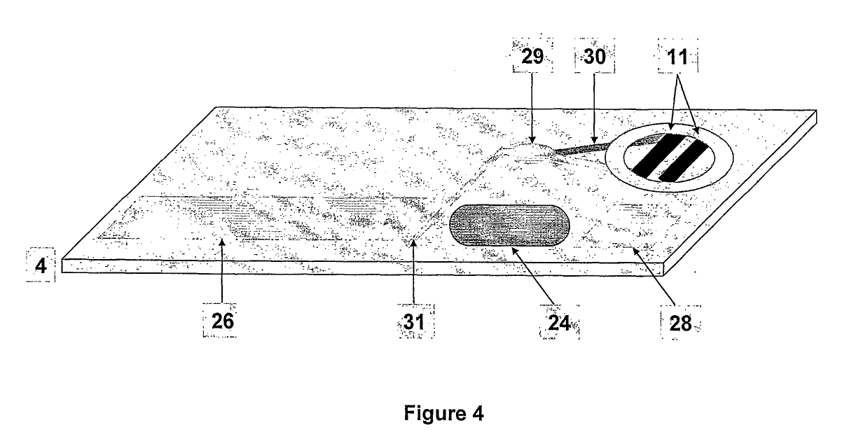

With specific reference to FIGS. 1-4, the invention is described in greater detail. In one embodiment of the invention, the self-contained apparatus 1 is formed from a plurality of layers, preferably three layers: a lower substrate 4, a middle substrate 3, and an upper substrate 2. Each layer may have components situate therein, such that when they layers are joined, the components are capable of performing the method of the invention. The apparatus may be about 2 inches by 3 inches by 0.05 inches, or may be as large as 5 inches by 7 inches by 0.25 inches. Preferably the apparatus is the size of a standard credit card. In thickness, the apparatus may range from about 0.05 inches to the thickness of a standard credit card to about a thickness of 0.25 inches. Preferably the apparatus is, in thickness, about 0.1 inches to about 0.25 inches.

In some embodiments, the apparatus may be constructed from two layers, an upper substrate and a lower substrate having components situate therein or embedded thereon, wherein the joining of the upper and lower substrates forms the various microchannels and microchambers of the apparatus by matching upper and lower surfaces of the microchannels to be formed.

Means of building such an apparatus may be drawn from methods known in the art, such as traditional lithography, soft lithography, and laminate technologies. Indeed, using the newer CAD based composite lamination techniques, the apparatus may be built up as a successive series of lamination layers, the design dictating the presence or absence of material such that the microchannels are formed. Such an embodiment of the invention would then comprise a single substrate comprising the components of the device embedded therein during its construction. Various components of the apparatus may then be inserted during the lamination process such that the completed device has all necessary components and microchannels.

In another embodiment of a three layer apparatus, the device is built with only the upper and lower substrates joined with a central space between the layers. The central substrate is prepared separately, and is adapted to be laterally inserted into the space between the two-layer construct. In this way, different central substrates may be manufactured which fit within a standardized two-layer construct capable of interfacing with a variety of such central substrates. The layers of the apparatus are generally sealed together sufficiently to form a mechanical seal, as well as preferably a thermal seal, the seals being sufficiently strong to withstand the stress of the apparatus being flexed during handling and normal use.

Components to be mounted, embedded, or situate in the constructed apparatus include, but are not limited to, a microneedle pad for piercing the skin of the subject and drawing a blood sample, an air pump bladder for creating the motive force to move the blood into blood entry channels, and at least one valve to insure one-way flow of the blood sample and the reagent-treated sample. Valves may be of any type known in the art which allow only one-way passage of fluid, such as that shown is published U.S. Patent Applications 2003/0197139A1 and 2005/0106066A1, specifically incorporated herein by reference in their entireties.

Blood has a viscosity approximately 3.6 times that of water, and may be forced into microchannels using different pump mechanisms, such as piezoelectric pumps, microsyringe pumps, electroosmotic pumps, and the like. While such pumps may be useful in the present invention, it is preferred to use simpler pump mechanisms such as gravity, capillary action, absorption by porous material, chemically induced pressure or vacuum, or manually driven bladder pumps such as that described herein and depicted in the Figures. Microfluidic channels, devices, and pumps are known in the art, such as those in U.S. Pat. Nos. 6,488,896B2, 5,932,100, and 6,136,272, previously incorporated herein in their entirety. Weigl, Advanced Drug Delivery Reviews, 55 (2003) 349-377, also previously incorporated herein, also discloses many such microfluidic components which may be adapted for use in the present invention.

Additional components may include a flat cell battery, a solar cell, or the like to power certain components of the apparatus (e.g., the logic chip and the display indicators), a variety of collection chambers and bulbs, adhesive seals and companion adhesion rings, backflow check valves, logic chips, validity indicator circuits, and microchannels to conduct fluid from the initial blood sample to the single cell capillaries which carry the cells to be counted into the blood cell counting channel. The apparatus may comprise fewer or additional components as necessary to practice the desired particular embodiment of the invention.

In one embodiment of the invention, a blood sample of between about 50 μl and 2 ml, preferably between about 100 μl and 1 ml, more preferably between 100 μl and 0.5 ml, more preferably between about 100 μl and 250 μl is drawn by the user by applying a portion of the body, such as a digit, to the operations button 9, a small area of the apparatus adapted to comprise a microneedle pad 16. Generally, the sample volume is sufficiently small to permit disposal by the end user within regulatory agency requirements. The exterior surface of the upper substrate 2 may have printed or etched thereupon instructions for use 8, and may have a clean sample indicator 7, which indicates that a clean sample has been introduced into the device. Applying the digit with sufficient pressure, the blood sample flows from the digit into the apparatus. In one embodiment, the microneedle pad 16 is surrounded with a concentric bladder 15 sealed to the external surface of the upper substrate with a bladder seal 13 and optionally with a bladder adhesion ring 14 which mates with the bladder seal. The bladder acts as a manually driven air pump which, upon the user's application of pressure to the needle pad, produces sufficient force to push the blood into the inner portions of the apparatus. In another embodiment, the bladder is located adjacent to, or beneath the microneedle pad. The pressure applied to the bladder 15 forceably evacuates the air held in the bladder through air suction channels 30 into an air accumulator bulb 29 connected to an air suction draw tube 31. Upon successive presses and releases of the bladder a motive force is generated which forces the blood sample through the blood entry channels 23 into the sample holding chamber 22, while generating vacuum in the air suction channel 30 upon the release phase of the press/release cycle. Additionally, the air suction draw tube connects to the end of the sample line, with a backflow check valve 25 amidst the draw tube to allow suction at the end of the sample line.

The bladder may be formed of flexible vinyl, or other material with sufficient flexibility and rigidity to operate successfully as an air pump bladder. In one embodiment, between 1 and 10 presses of the bladder are required to move the blood sample through the apparatus and achieve a successful count, preferably 1-5 presses and releases, and most preferably 1-3 presses and releases.

In an alternative embodiment, the air in the bladder may be evacuated through a one-way valve open to the exterior of the device provided the blood sample is propelled into the blood entry channels 23 and then into the sample holding chamber 22.

A sampling check valve 21 may be positioned adjacent to the sample holding chamber 22 to allow one-way flow from the sample holding chamber 22 into the blood and reagent mixing bulb 20. Reagents are either already present in the reagent mixing bulb or are moved into the reagent mixing bulb from reagent holding chambers via the same force applied from the bladder, such that as the blood sample enters the reagent mixing bulb, it is mixed with the reagents. These reagents dilute the sample to achieve the proper viscosity and composition for the blood cells to flow into the capillary like blood cell counting channels 17 in a later stage of the apparatus and method. The reagents may also accomplish such tasks as cleaning the sample, and removing platelets, serum proteins, and additional whole blood components other than the red and white blood cells.

The blood and reagent mixing bulb passes the now treated sample to a pair of blood cell filtration elements 19 comprising filters such as for example nanofilters designed for filtering red and white blood cells. Passage from the blood and reagent mixing bulb into the pair of blood cell filtration elements may also trigger a signal from the logic chip 27 to illuminate the clean sample indicator 7. In one embodiment, one of the pair of filtration elements allows only white blood cells to pass, while the other allows only red cells to pass. The filtration elements may accomplish this task by any means known in the art, such as size filtration, adhesion, antibodies directed to the respective cell types, color, orientation, texture, or the like. The initial sample is thereby split in two; one portion passing through a filtration element which allows only red cells to pass, while the other portion passing through a filtration element which allows only white cells to pass. The reagent mixing bulb may itself provide the splitting of the sample into two fractions by having two chambers, each of which provides the reagents necessary for filtering the desired cell type. Alternatively, the reagent mixing bulb may comprise a single chamber which adds reagents and yields a treated sample, which treated sample then splits into each of the pair of filtration elements. The filtration elements are then provided with the means of filtering the desired blood cell type. Each of the pair of filtration elements passes their respective treated samples to one of a pair of blood cell collection chambers 18. Each of these chambers passes their respective samples into blood cell counting channels 17, which may be sized to allow only a single file line of cells to occupy the channels.

The sample holding chamber 22 allows for pressure to build as the bladder is pressed and released, releasing the blood sample through the sampling check valve 21 and into the blood and reagent mixing bulb 20 once sufficient pressure has been achieved. This insures that the blood sample is of sufficient volume, which insures that the blood and reagent mixing bulb 20 is filled with a specific fixed volume of blood sample. The series of chambers and channels are adapted to provide homogenous treated samples to the blood cell counting channels 17, which can contain a specific fixed volume of treated sample. The volume of treated sample reaching the counting channels is fixed while the number of cells present therein is directly proportional to the concentration of blood cells in the original sample, thus, the number of cells reaching the blood cell counting channels provide a direct measurement of the number of cells present in the original blood sample. That is, so long as the initial blood sample is of sufficient volume and the bladder has been pumped to sufficient pressure, a fixed volume of homogenous treated sample reaches the blood cell counting chambers. The blood cell counting chambers 17 will only reach full capacity if the blood sample was of sufficient volume, and if the pressure achieved through pumping the bladder was also sufficient. In one embodiment, the apparatus has a validity indicator circuit 28 which indicates that the blood cell counting chambers are full with an LED or other such indicator (e.g., LCD, backlit LCD, surface gas lamp, and the like) on the exterior surface of the upper substrate. In one embodiment the LED is readable in bright or dim light at a distance of about 1-3 feet, allowing hand-held readability.

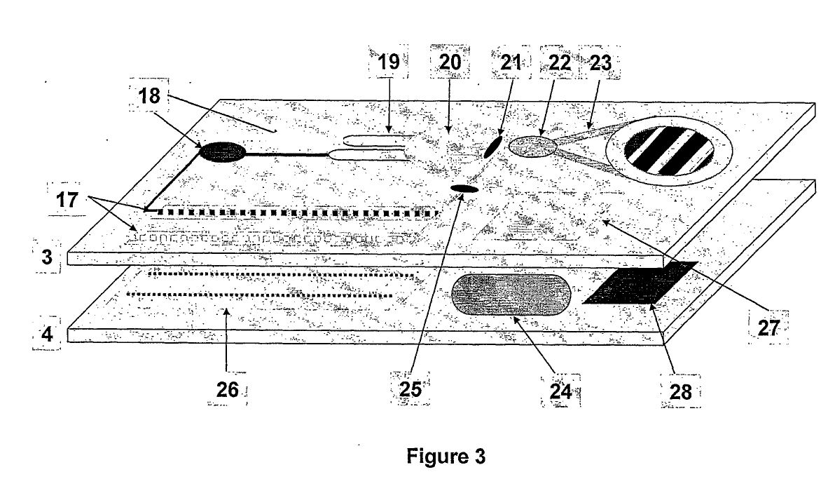

As shown in FIG. 3, the distal portion of the blood cell counting channels is positioned adjacent to cell counting sensors, which may be for example a linear array of charge-coupled devices (CCDs) 26. The CCDs are powered by a power source, such as a flat cell battery 24, or a solar cell embedded in the exterior surface of the upper substrate, or the like, which CCDs pass their signal to a logic chip 27 also powered by the battery, solar cell, or the like, in operational control of a validity indicator circuit 28.

Once the treated samples of white and red blood cells, respectively, have entered the blood cell counting channels 17, the blood cells contained therein fill the end of the channels, which may narrow to a size too small to allow the cells to move any further, thus causing the blood cell counting channels to fill with cells. The narrowed ends of the blood cell counting channels connect to the air suction draw tube 31, which provides suction to keep the counting channels filling, until their full capacity is reached.

The blood cell counting channels 17 are filled with a fixed volume of treated sample, but only partially filled with red or white blood cells, respectively, and a measure of the extent to which they are full with cells provides a measure of the number of cells present in the sample. Because the amount of the treated blood sample reaching the blood cell counting channels is a fixed volume, independent of the actual volume of the initial blood sample, the subject's blood cell count can be measured by determining how full of cells the blood cell counting chambers are. The logic chip 27 is programmed to determine the count of blood cells by employing the counting sensors to detect the level of fullness of the blood cell counting channels. The logic chip 27 then carries out the operation of comparing the blood cell count determined using the CCDs to a standardized range of numbers for healthy subjects, thereby determining whether the initial blood sample falls within or outside normal healthy ranges.

In another embodiment, the cell counting channels 17 may be sized to allow more than a single file line of cells to occupy the channel. In such an embodiment, the cell counting may be accomplished by means of a two-dimensional array of sensors or by means of one or more density measurements using, for example, a beam of light to measure transmittance and/or absorbance through the sample. The logic chip 27 is programmed to determine the count of blood cells by employing the two-dimensional array of counting sensors or the density measurements, and carries out the operation of comparing the blood cell count to a standardized range of numbers for healthy subjects, thereby determining whether the initial blood sample falls within or outside normal healthy ranges.

In one embodiment, the logic chip signals the white cell count indicator 5 and the red cell count indicator 6 to display the resulting respective counts. The exterior surface of the upper substrate may have etched or printed thereupon a scale showing minimum and maximum blood cell counts at opposed ends of each of the indicators. The indicators themselves may be linear arrays of LEDs, of the same or different color. Other embodiments may use LCDs, surface gas lamps, or any other signaling mechanism capable of indicating a graduated scale of results. In one embodiment, the scales on the exterior surface of the upper substrate may have only a few regions, such as “below normal,” “normal,” and “above normal.” Alternatively, the scales may be more finely graded, or may have actual quantity measurements listed as number of cells per ml. In the latter case, a printed or etched key on the exterior surface of the lower substrate, or printed on packaging material, may describe the normal ranges and variances therefrom.

The normal range varies but is generally considered to be between about 4.3-10.8×10⁹ cells per liter. White blood cells comprise a variety of blood cell types, including granulocytes, lymphocytes, monocytes, eosinophils, and basophils. Red blood cells, or erythrocytes, are normally found in a range of between about 4.2-5.9×10¹² cells per liter. The apparatus may display actual counts in terms of cells per ml, cells per liter, either of which may have an accuracy of within about +/−2-5%, or in the broad ranges described above, wherein an accuracy of between about +/−10% is generally sufficient.

In one embodiment, the initial sample from the patient is a volume of blood sufficient to provide accurate counts of cells within the desired accuracy range. In some applications, an accuracy range of about +/−10% is generally sufficient, but in other applications, a higher accuracy range of about +/−5% or as low as about +/−2.5% may be desirable. The number of cells actually sampled by the invention may be as high as about 100,000 cells, although accurate results may be obtained with a smaller sample number on the order of about 50,000 cells, or 25,000 cells, or 10,000 cells, or 5,000 cells.

The device of the invention provides sufficiently accurate results upon repeated uses, such that results generally will fall within about +/−10%, or +/−5%, or +/−2.5% on repeated samplings from a patient.

### Red Blood Cell Counter

In one embodiment of the invention, an apparatus and method are provided which count red blood cells. Instead of a pair of channels conducting the flow of the blood sample to the blood cell counting channels, in this embodiment only a single channel is employed. This channel narrows in the same fashion as the red blood cell channel in the embodiment counting both red and white blood cells, however, white blood cells are simply collected in a white blood cell collection device, filtered out of the flow via a filter based on size, or removed from the sample during mixing in the blood and reagent mixing bulb 20 by reagents adapted to lyse white blood cells. Thus, the only blood cells reaching the blood cell counting channel are red blood cells. This embodiment thus allows for the counting of red blood cells only.

### White Blood Cell Counter

In one embodiment of the invention, an apparatus and method are provided which count white blood cells. Instead of a pair of channels conducting the flow of the blood sample to the blood cell counting channels, in this embodiment only a single channel is employed. This channel narrows in the same fashion as the white blood cell channel in the embodiment counting both red and white blood cells, however, red blood cells are simply collected in a red blood cell collection device, or removed from the sample during mixing in the blood and reagent mixing bulb 20 by reagents adapted to lyse red blood cells. Thus, the only blood cells reaching the blood cell counting channel are white blood cells. This embodiment thus allows for the counting of white blood cells only.

### Microchannels and Microchambers

The microchannels of the invention may range in diameter from about 5 mm in the proximal portions of the apparatus near the microneedle pad, to as small as 3-5 μm at the distal ends of the counting channels. A normal red blood cell is approximately 6-8 μm, thus the distal end of the red cell counting channel is 5 μm or smaller to prevent escape of the red blood cells. A normal white blood cell may range in size from about 10-16 μm, thus the distal end of the white cell counting channel is less than 10 μm, but may be as small as 5 μm or smaller to prevent escape of the white blood cells. As an alternative to the narrowing of the distal ends of the cell counting channels, appropriate microfilters may be positioned at such distal ends with pore sizes smaller than the size of the respective blood cells, thereby preventing the escape of the blood cells. Thus, as used herein, the “narrowing” of the cell counting channels is also intended to contemplate such microfilters at the distal ends of the cell counting channels.

The microchamber of the apparatus has a variety of capacities dependent on the particular component's function in the apparatus. Thus, the sample holding chamber 22 may hold between about 50 μl and about 1 ml of blood sample, the blood and reagent mixing bulb 20 may hold between about 100 μl and 1 ml of blood-reagent mixture, and the blood cell collection chambers 18 may hold between about 10 μl and 500 μl.

In one embodiment of the invention, the apparatus indicators display results comprising at least one of total cell count, white blood cell count, and red cell count. Other embodiments display results comprising at least two such counts, or all three.

### EXAMPLES

The present invention will be further understood by reference to the following non-limiting examples.

### Example 1

An apparatus is built as depicted in FIGS. 1-4. The apparatus has a scale printed next to the white and red cell count indicators having three ranges—below normal, normal, and above normal. A healthy subject with a normal blood cell count applies her thumb to the microneedle pad 16, and in accordance with the instructions 8 printed on the exterior adjacent to the microneedle pad 16, presses and releases her thumb five times slowly. Within about one minute, the clean sample indicator 7 illuminates followed by the validity indicator circuit, indicating that the blood cell counting channels are full with treated samples containing cells. Within about 45 seconds to about 2 minutes, both the white 5 and red 6 cell count indicators illuminate in the center of the normal range, indicating that the subject's blood counts are normal.

### Example 2

An unused apparatus as described in Example 1 is used by a subject suffering from an infection. The subject performs the same steps as the subject in Example 1A. The clean sample indicator illuminates, followed by the validity indicator The red cell count indicator illuminates in the normal range, but the white cell count indicator illuminates in the “above normal” range printed on the exterior surface of the upper substrate.

### Example 3

An apparatus similar to that in Example 1 is constructed with finer gradations printed on the exterior surface of the upper substrate, with five ranges: far below normal, below normal, normal, above normal, far above normal. The subject is undergoing chemotherapy following discharge from a hospital a week after removal of a malignant tumor. The subject is instructed by a doctor to use the apparatus of the invention once a week to check her blood cell counts, and to come to the hospital if the white cell count drops into the far below normal range for additional medical intervention. The subject performs the same steps as the subject in Example 1. The clean sample indicator illuminates, followed by the validity indicator. The red cell count indicator illuminates in the below normal range, but the white cell count indicator illuminates in the “far below normal” range. The subject then proceeds to the hospital for treatment, or contacts her physician for further advice.

All references cited herein are expressly incorporated by reference herein in their entireties. A number of embodiments of the invention have been described. It will be understood that various modifications may be made to the disclosed embodiments without departing from the spirit and scope of the invention, including the addition of other features to the apparatus. Accordingly, other embodiments are intended to be within the scope of the following claims.

I claim:

1. A self-contained apparatus for counting blood cells comprising a substrate with an upper surface and a lower surface, the substrate comprising;
(a) a microneedle pad, the microneedle pad being capable of drawing a blood sample from the digit of a human subject;
(b) a pump capable of forcing the blood sample into a blood and reagent mixing bulb, wherein the blood sample is treated with reagents to yield a treated sample free of undesired blood components, and of desired viscosity and dilution;
(c) blood cell filtration elements to filter white blood cells into a white blood cell counting channel and red blood cells into a red blood cell counting channel, the diameter of at least the distal portion of the channels being sufficient for only a single file of blood cells to occupy the counting channels, wherein the distal ends of the channels narrow further to prevent the further passage of blood cells; and
(d) an array of counting sensors adjacent to the blood cell counting channels, the sensors being in communication with a logic chip adapted to determine blood cell counts from the data communicated by the sensors and to display the blood cell counts on cell count indicators on the upper surface of the substrate.

2. The apparatus of claim 1, further comprising at least one component selected from the group consisting of a validity indicator circuit, a clean sample indicator, a bladder seal, a bladder adhesion ring, a sample holding chamber, a flat cell battery, a solar cell, a blackflow check valve, an air accumulator bulb, air suction channels, an air suction draw tube, an air entry channel, and instructions printed or etched on the upper surface of the substrate.

3. The apparatus of claim 1, wherein the substrate comprises an upper substrate and a lower substrate, wherein the joinder of the upper and lower substrates forms the channels and chambers by complementary grooves in an interior surface of the upper and lower substrates.

4. The apparatus of claim 1, wherein the substrate comprises a plurality of layers laid down by thin layer deposition under computer aided design control.

5. The apparatus of claim 1, wherein the substrate comprises an upper substrate, a middle substrate, and a lower substrate joined together.

6. The apparatus of claim 1, wherein the pump is manually operated by the subject successively pressing and releasing the microneedle pad.

7. The apparatus of claim 1, wherein the blood sample drawn into the apparatus is of volume between about 50 μl and about 1 ml.

8. The apparatus of claim 1, wherein the blood cell counts are displayed on the upper surface of the substrate by LEDs.

9. A self-contained apparatus for counting red blood cells comprising a substrate with an upper surface and a lower surface, the substrate comprising;
(a) a microneedle pad, the microneedle pad being capable of drawing a blood sample from the digit of a human subject;
(b) a pump capable of forcing the blood sample into a blood and reagent mixing bulb, wherein the blood sample is treated with reagents to yield a treated sample free of undesired blood components, and of desired viscosity and dilution;
(c) a blood cell filtration elements to filter red blood cells into a red blood cell counting channel, the diameter of at least the distal portion of the channel being sufficient for only a single file of blood cells to occupy the counting channel, wherein the distal end of the channel narrows to prevent the further passage of blood cells; and
(d) an array of counting sensors adjacent to the blood cell counting channel, the sensors being in communication with a logic chip adapted to determine a blood cell count from the data communicated by the sensors and to display the blood cell count on cell count indicators on the upper surface of the substrate.

10. A self-contained apparatus for counting white blood cells comprising a substrate with an upper surface and a lower surface, the substrate comprising;
(a) a microneedle pad, the microneedle pad being capable of drawing a blood sample from the digit of a human subject;
(b) a pump capable of forcing the blood sample into a blood and reagent mixing bulb, wherein the blood sample is treated with reagents to yield a treated sample free of undesired blood components, and of desired viscosity and dilution;
(c) a blood cell filtration elements to filter white blood cells into a white blood cell counting channel, the diameter of at least the distal portion of the channel being sufficient for only a single file of blood cells to occupy the counting channel, wherein the distal end of the channel narrows to prevent the further passage of blood cells; and
(d) an array of counting sensors adjacent to the blood cell counting channel, the sensors being in communication with a logic chip adapted to determine a blood cell count from the data communicated by the sensors and to display the blood cell count on cell count indicators on the upper surface of the substrate.

11. A method of counting blood cells in a subject comprising the steps of:
(a) pressing and releasing a microneedle pad of an apparatus of any of claims 1-10; and
(b) reading the result on an upper surface of the apparatus.
